(19) United States
(12) Patent Application Publication
(10) Pub. No.: US 2025/0017573 A1
(43) Pub. Date: Jan. 16, 2025

(54) FEMALE LIFE STAGE PREDICTION METHOD, FEMALE LIFE STAGE PREDICTION PROGRAM, AND FEMALE LIFE STAGE PREDICTION SYSTEM

(71) Applicant: TOTO LTD., Fukuoka (JP)

(72) Inventors: Mizuki WATANABE, Fukuoka (JP); Yuumi HAYASHI, Fukuoka (JP)

(21) Appl. No.: 18/897,643

(22) Filed: Sep. 26, 2024

Related U.S. Application Data
(63) Continuation of application No. PCT/JP2023/011761, filed on Mar. 24, 2023.

(30) Foreign Application Priority Data
Apr. 20, 2022 (JP) 2022-069515

(51) Int. Cl. A61B 10/00; G01N 33/74

(52) U.S. Cl. CPC A61B 10/0012; G01N 33/74

(57) ABSTRACT

A female life stage prediction method includes measuring a secretion amount of a hormone of a user, based on at least any of bodily fluid discharged to an outside of a body of the user or a volatile component of the bodily fluid, recording the secretion amount measured, estimating a fluctuation in the secretion amount of the hormone of the user, based on the secretion amount recorded, making a prediction about a time of at least any of menarche, menstruation, or menopause of the user, based on the fluctuation estimated, and issuing a notification of a result of the prediction.

### CROSS-REFERENCE TO RELATED APPLICATIONS

This application is a bypass continuation of PCT Application No. PCT/JP2023/011761 filed on Mar. 24, 2023, which claims pf priority to Japanese Patent Application No. 2022-069515, filed on Apr. 20, 2022; the entire contents of which are hereby incorporated by reference.

### FIELD

Aspects of the present invention generally relate to a female life stage prediction method, a female life stage prediction program, and a female life stage prediction system.

### BACKGROUND

During life, women go through life stages, experiencing menarche (first menstruation) in adolescence, repeated menstruation from adolescence to sexual maturation, and then menopause in which menstruation ends. The onset of menarche varies among individuals and is difficult to predict based on daily life. Thus, the individual may not be prepared for menarche (may not have sanitary products prepared or may not be mentally ready). Further, while menstruation occurs in regular cycles, the cycle may be late or early, for example. Being able to predict the times of menarche and menstruation can be useful for health care management and activities of women. The onset of menopause also varies among individuals, and predicting menopause would also be useful for health care management and activities of women.

The present invention has been made in recognition of such problems, and an object of the present invention is to provide a female life stage prediction method, a female life stage prediction program, and a female life stage prediction system in which the time of at least any of menarche, menstruation, or menopause can be predicted.

### SUMMARY

A female life stage prediction method according to a first aspect of the present invention includes: measuring a secretion amount of a hormone of a user, based on at least any of bodily fluid discharged to an outside of a body of the user or a volatile component of the bodily fluid; recording the secretion amount measured; estimating a fluctuation in the secretion amount of the hormone of the user, based on the secretion amount recorded; making a prediction about a time of at least any of menarche, menstruation, or menopause of the user, based on the fluctuation estimated; and issuing a notification of a result of the prediction.

According to this female life stage prediction method, it is possible to estimate, from discharge from a human body, fluctuation in the secretion amount of the hormone and to predict the time of at least any of menarche, menstruation, or menopause from the result. This makes it possible to prepare for menarche or menstruation, for example. Alternatively, this makes it possible to prepare for symptoms of menopause.

A female life stage prediction system according to a second aspect of the present invention includes: a measurement unit configured to measure a secretion amount of a hormone of a user, based on at least any of bodily fluid discharged to an outside of a body of the user or a volatile component of the bodily fluid; a recording unit configured to record the secretion amount measured; a hormone secretion amount fluctuation estimation unit configured to estimate a fluctuation in the secretion amount of the hormone of the user, based on the secretion amount recorded; a life stage prediction unit configured to make a prediction about a time of at least any of menarche, menstruation, or menopause of the user, based on the fluctuation estimated; and an output unit configured to output a result of the prediction.

According to this female life stage prediction system, it is possible to estimate, from discharge from a human body, fluctuation in the secretion amount of the hormone and to predict the time of at least any of menarche, menstruation, or menopause from the result. This makes it possible to prepare for menarche or menstruation, for example. Alternatively, this makes it possible to prepare for symptoms of menopause.

According to a third aspect of the present invention, in the female life stage prediction system of the second aspect, the bodily fluid is sweat, and the measurement unit includes a detection unit installed on a seat face of a toilet seat and configured to detect the hormone contained in the sweat or in a volatile component of the sweat.

According to this female life stage prediction system, the secretion amount of the hormone can be measured simultaneously with the daily act of sitting on a toilet seat. Thus, there is no need to prepare a tool such as a pregnancy test kit, and the time and effort required for measurement is reduced.

According to a fourth aspect of the present invention, in the female life stage prediction system of the second aspect, the bodily fluid is urine, and the measurement unit includes a detection unit configured to detect, from air in an inner toilet bowl, the hormone contained in a volatile component of the urine.

According to this female life stage prediction system, the secretion amount of the hormone can be measured simultaneously with the daily act of sitting on a toilet seat and excreting. Thus, there is no need to prepare a tool such as a pregnancy test kit, and the time and effort required for measurement is reduced.

According to a fifth aspect of the present invention, the female life stage prediction system of the third or fourth aspect further includes: a seating sensor configured to detect a person sitting on a toilet seat, in which the measurement unit starts measurement when the seating sensor detects that the user is seated on the toilet seat.

According to this female life stage prediction system, the hormone secretion amount can be automatically measured by performing the daily act of sitting on a toilet seat. Since the secretion amount can be recorded every day without forgetting, accuracy of estimation of the fluctuation in the secretion amount and accuracy of life stage prediction based on the estimated fluctuation can be improved, for example.

According to a sixth aspect of the present invention, in the female life stage prediction system of the second aspect, the life stage prediction unit predicts an onset of menarche when the fluctuation of the hormone estimated satisfies a predetermined condition.

According to this female life stage prediction system, the onset of menarche can be predicted, and thus the user can prepare. For example, sanitary products can be prepared before the user's first menstruation and advice can be given in advance on how to deal with menstruation, including how to use the sanitary products. As a result, menarche can be experienced with less worry.

According to a seventh aspect of the present invention, in the female life stage prediction system of the sixth aspect, the life stage prediction unit predicts a time of next menstruation, based on the fluctuation of the hormone estimated, the fluctuation being cyclical.

According to this female life stage prediction system, since the fluctuation in the secretion of the hormone changes so as to cyclically fluctuate after menarche, it is possible to predict the time of menstruation, prepare sanitary products, and prepare for health problems in advance.

According to an eighth aspect of the present invention, the female life stage prediction system of the seventh aspect further includes: an imaging unit configured to capture an image of a surface of an inner toilet bowl, in which the life stage prediction unit predicts a time of menstruation, based on a time of actual menstruation determined based on the image captured by the imaging unit and the cyclical fluctuation of the hormone estimated.

According to this female life stage prediction system, by confirming the time of actual menstruation on the basis of the image captured by the imaging unit, prediction accuracy of the time of future menstruation can be improved.

According to a ninth aspect of the present invention, the female life stage prediction system of any one of the second to fourth aspects further includes: a health state prediction unit configured to calculate a stress level of the user, in which the stress level is stored in association with the secretion amount of the hormone measured when the stress level is calculated, and the health state prediction unit predicts an onset of premenstrual syndrome, based on a relationship between the stress level stored and the secretion amount of the hormone associated with the stress level, and a current secretion amount of the hormone.

According to this female life stage prediction system, it is possible to predict health problems (e.g., premenstrual syndrome) of the user before menstruation from the latest measurement result on the basis of the relationship between the hormone and the stress level in the past.

According to a tenth aspect of the present invention, the female life stage prediction system of any one of the second to fourth aspects further includes: a sensor configured to acquire blood flow information of the user; and a health state prediction unit configured to predict a possibility of anemia of the user, based on the blood flow information and the fluctuation of the hormone estimated.

According to this female life stage prediction system, it is possible to accurately predict the possibility of anemia by taking into account both the blood flow information and the secretion amount of the hormone.

According to an eleventh aspect of the present invention, in the female life stage prediction system of any one of the second to fourth aspects, the life stage prediction unit predicts that menopause is approaching, based on a decreasing trend of the secretion amount of the hormone.

According to this female life stage prediction system, it is possible to predict that menopause is approaching. Accordingly, for example, the possibility of the occurrence of menopausal symptoms can be predicted, which can be useful for health care management and other purposes.

According to a twelfth aspect of the present invention, the female life stage prediction system of any one of the second to fourth aspects further includes: a controller configured to suggest an action to the user, based on the time of menstruation predicted and calendar information.

According to this female life stage prediction system, the prediction result of the time of menstruation can be useful in scheduling. For example, the prediction result of the time of menstruation can be used to plan vacation days and schedule travel and other activities.

According to a thirteenth aspect of the present invention, in the female life stage prediction system of any one of the second to fourth aspects, a fluctuation state of the secretion amount of the hormone of the user is transmitted to a server, and the server makes a comparison between the fluctuation state and a hormone secretion amount fluctuation model, and outputs menstrual state information of the user based on a result of the comparison.

According to this female life stage prediction system, the user can grasp the result of comparison between the fluctuation in the secretion amount of her own hormone and that of a typical hormone secretion amount fluctuation model. For example, the user can grasp a difference such as whether her own cycle of menstruation is long or short compared with a typical cycle. This makes it possible to, for example, provide advice, such as seeking consultation with a doctor, and reference information to the user.

A female life stage prediction program according to a fourteenth aspect of the present invention causes a computer to execute processing including: estimating, based on a secretion amount of a hormone of a user measured based on at least any of bodily fluid discharged to an outside of a body of the user or a volatile component of the bodily fluid, and stored in a recording unit, a fluctuation in the secretion amount of the hormone of the user; making a prediction about a time of at least any of menarche, menstruation, or menopause of the user, based on the fluctuation estimated; and outputting a result of the prediction.

According to this female life stage prediction program, it is possible to estimate, from discharge from a human body, fluctuation in the secretion amount of the hormone and to predict the time of at least any of menarche, menstruation, or menopause from the result. This makes it possible to prepare for menarche or menstruation, for example.

According to aspects of the present invention, provided are a female life stage prediction method, a female life stage prediction program, and a female life stage prediction system in which the time of at least any of menarche, menstruation, or menopause can be predicted.

### BRIEF DESCRIPTION OF THE DRAWINGS

FIG. 1 is a schematic block diagram illustrating a female life stage prediction system according to an embodiment;

FIG. 2(a) and FIG. 2(b) are schematic views for explaining part of the female life stage prediction system according to the embodiment;

FIG. 3 is a plan view for explaining part of the female life stage prediction system according to the embodiment;

FIG. 4 is a flowchart illustrating an example of a female life stage prediction method of the female life stage prediction system according to the embodiment;

FIG. 5 is a flowchart illustrating an example of the female life stage prediction method of the female life stage prediction system according to the embodiment;

FIG. 6 is a flowchart illustrating an example of the female life stage prediction method of the female life stage prediction system according to the embodiment;

FIG. 7 is a flowchart illustrating an example of the female life stage prediction method of the female life stage prediction system according to the embodiment;

FIG. 8 is a flowchart illustrating an example of the female life stage prediction method of the female life stage prediction system according to the embodiment;

FIG. 9 is a flowchart illustrating an example of the female life stage prediction method of the female life stage prediction system according to the embodiment;

FIG. 10 is a flowchart illustrating an example of the female life stage prediction method of the female life stage prediction system according to the embodiment; and

FIG. 11 is a schematic block diagram illustrating another female life stage prediction system according to the embodiment.

### DETAILED DESCRIPTION

According to aspects of the present invention, provided are a female life stage prediction method, a female life stage prediction program, and a female life stage prediction system in which the time of at least any of menarche, menstruation, or menopause can be predicted.

Hereinafter, embodiments of the present invention will be described with reference to the drawings. Note that, in the drawings, similar components are denoted by like reference signs, and detailed description thereof will be omitted as appropriate.

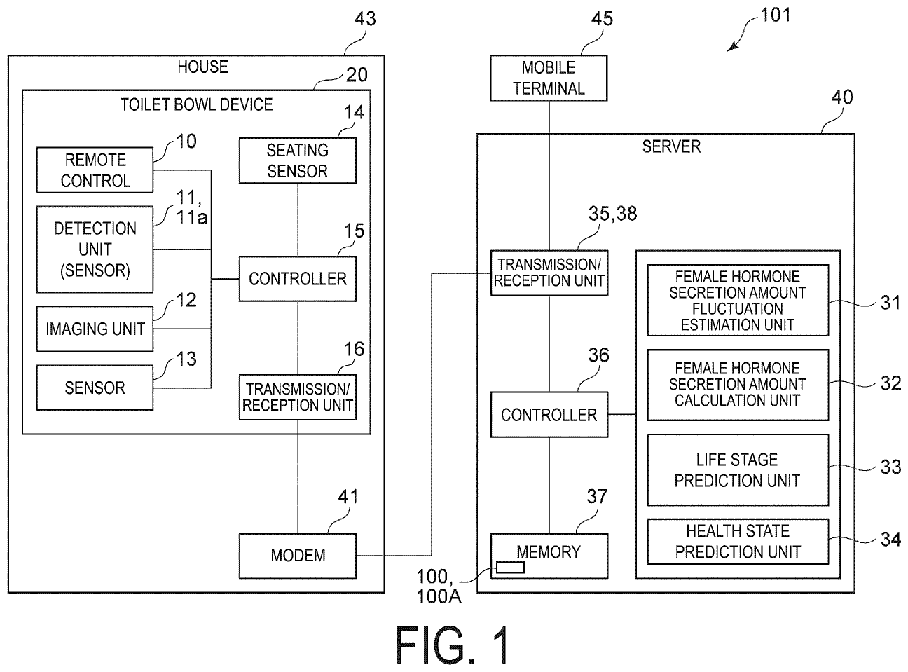

FIG. 1 is a schematic block diagram illustrating a female life stage prediction system according to an embodiment.

A female life stage prediction system 101 that executes a female life stage prediction method according to the embodiment includes a measurement unit 11 that measures a female hormone of a user, a female hormone secretion amount fluctuation estimation unit 31, a life stage prediction unit 33, a transmission/reception unit 35 (output unit 38), and a memory 37 (recording unit).

In the embodiment, the measurement unit 11 measures the secretion amount of a female hormone of the user. The memory 37 records the secretion amount measured by the measurement unit 11. The female hormone secretion amount fluctuation estimation unit 31 estimates a fluctuation in the secretion amount of the female hormone of the user on the basis of the secretion amount recorded in the memory 37. The life stage prediction unit 33 predicts a female life stage of the user on the basis of the fluctuation estimated by the female hormone secretion amount fluctuation estimation unit 31. The output unit 38 outputs a prediction result of the female life stage predicted by the life stage prediction unit 33 to an external unit or the like. Thus, the user is notified of the prediction result of the female life stage.

Note that the term “female life stage” refers to, for example, the time of experiencing menarche (adolescence), the time of repeated menstruation (maturation), or the time of reaching climacteric (menopause). The female life stage predicted by the female life stage prediction system 101 includes at least any of the time of menarche, the time of menstruation, the time of climacteric, or the time of menopause. Here, the time may include at least any of the start time or the end time. The time of menstruation predicted by the female life stage prediction system 101 may be the menstrual cycle.

Prediction of the time may include predicting that the time is close. For example, the female life stage prediction system 101 predicts that the time will arrive in the near future (or future). The near future or future may be, for example, a time about one day to two weeks away from the present, but no limitation is intended. Further, in this specification, the term “menstruation” alone refers to menstruation after menarche.

As illustrated in FIG. 1, at least part of the measurement unit 11 is provided in, for example, a toilet bowl device 20 (toilet device) installed in a building such as a house 43 of the user. The toilet bowl device 20 may be part of the female life stage prediction system 101. In this example, the toilet bowl device 20 further includes a remote control 10, an imaging unit 12, a sensor 13 for measuring blood flow information, a seating sensor 14, a controller 15, and a transmission/reception unit 16.

Note that the female life stage prediction system 101 according to the embodiment need not necessarily be installed in the toilet bowl device 20. For example, part of the female life stage prediction system 101 (a detection unit 11a, the sensor 13 that measures blood flow information, and the like) may be installed in a wash stand or the like.

The toilet bowl device 20 has, for example, a function of spouting wash water toward a private part of the user to wash the private part, and a deodorizing function of deodorizing air in the toilet bowl. The toilet bowl device 20 may have an automatic opening/closing function of automatically opening and closing the toilet seat and the toilet lid, a heating function of heating the toilet seat, and the like.

The remote control 10 includes a switch (e.g., a push button). The remote control 10 may include a touch panel or a display. The user can operate each function of the toilet bowl device 20 by operating the switch of the remote control 10. Specifically, a signal corresponding to the operation by the user is transmitted from the remote control 10 to the controller 15. The controller 15 controls the operation of each unit of the toilet bowl device 20 in response to the signal from the remote control 10.

The remote control 10 may include a switch for operating a function of the female life stage prediction system 101. For example, measurement in the female life stage prediction system 101 may be executed by an operation of the remote control 10.

The measurement unit 11 measures the secretion amount of a female hormone of the user on the basis of bodily fluid or the like (at least any of bodily fluid discharged to the outside of the body of the user or a volatile component of the bodily fluid) of the user. The bodily fluid discharged to the outside of the body is, for example, sweat or urine. Note that “outside of the body” may include the inside of the oral cavity. In this case, the bodily fluid discharged to the outside of the body may be, for example, saliva. The volatile component is a gas resulted from volatilization of the bodily fluid described above and includes, for example, an odor of the bodily fluid. The female hormone is not particularly limited to a specific hormone. As an example, the female hormone is estrogen or progesterone.

The secretion amount of the female hormone of the user measured by the measurement unit 11 may be an index value indicating the secretion amount of the female hormone of the user. For example, the secretion amount (measured value) of the female hormone measured by the measurement unit 11 may be the amount of the female hormone contained in the measured bodily fluid or the like, or the concentration of the female hormone contained in the measured bodily fluid or the like. Alternatively, the secretion amount (measured value) of the female hormone measured by the measurement unit 11 may be a value estimated from a physical quantity reflecting the secretion amount of the female hormone.

The measurement unit 11 includes the detection unit 11a (sensor) provided in the toilet bowl device 20. The detection unit 11a comes into contact with the bodily fluid or the like to detect the female hormone contained in the bodily fluid or the like. As an example, the detection unit 11a includes an element, an electrical state (e.g., resistance) of which changes when the element is bound to molecules of a specific female hormone. Accordingly, the detection unit 11a detects an electric signal (voltage value, current value, or the like) that changes according to the secretion amount of the female hormone contained in the bodily fluid or the like, and outputs a detected value corresponding to the detection result to the controller 15.

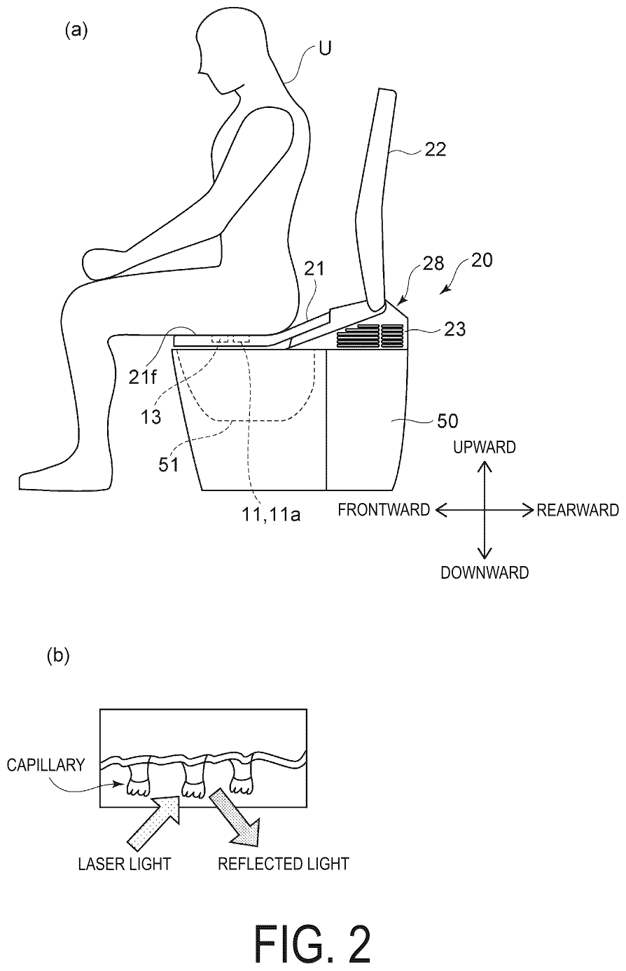

The imaging unit 12 can capture an image of the inside of an inner toilet bowl 51 (refer to FIG. 2(a)) of a toilet bowl 50. For example, the imaging unit 12 is an imaging sensor (camera) that captures an image of the surface of the inner toilet bowl 51 to acquire an image of the surface of the inner toilet bowl 51. The imaging unit 12 outputs the acquired image to the controller 15.

The sensor 13 is a sensor that can acquire blood flow information of the user. The blood flow information that can be acquired by the sensor 13 is, for example, information related to the blood flow rate and the pulse wave. The blood flow information that can be acquired by the sensor 13 may be information related to a component in the blood, such as an estimated value of the hemoglobin amount in the blood, for example. The blood flow information that can be acquired by the sensor 13 may be estimated from a measurement result of a physical quantity reflecting the blood flow information. The sensor 13 outputs the acquired blood flow information to the controller 15.

The seating sensor 14 detects a person sitting on a toilet seat 21 (refer to FIG. 2(a)). The seating sensor 14 is a sensor that can detect whether the user has been seated or is being seated on the toilet seat 21. As the seating sensor 14, for example, a distance measurement sensor of an infrared light emitting/receiving type can be used. The seating sensor 14 may be, for example, a microwave sensor or a pyroelectric sensor. Further, the seating sensor 14 may be, for example, a mechanical switch, an optical sensor, or a magnetic sensor that detects movement of the toilet seat, a capacitance sensor that detects a change in capacitance caused by seating, or a piezoelectric sensor that detects a change in pressure caused by seating. The seating sensor 14 may detect a user present above the toilet seat 21 immediately before the user is seated on the toilet seat 21. The seating sensor 14 outputs a detection result to the controller 15.

The controller 15 is communicably connected to the remote control 10, the detection unit 11a, the imaging unit 12, the sensor 13, the seating sensor 14, and the transmission/reception unit 16. An electric circuit such as a computer is used for the controller 15. The controller 15 includes, for example, a central processing unit (CPU). The controller 15 is, for example, a control circuit including a microcomputer.

The controller 15 transmits a command signal to each of the detection unit 11a, the imaging unit 12, the sensor 13, and the seating sensor 14 to control the operation of each of the detection unit 11a, the imaging unit 12, the sensor 13, and the seating sensor 14.

For example, upon receipt of a detection result from the seating sensor 14 indicating that the user has been seated on the toilet seat 21, the controller 15 causes the detection unit 11a to detect the female hormone, causes the imaging unit 12 to acquire an image, and causes the sensor 13 to acquire the blood flow information. Alternatively, the controller 15 may control the detection unit 11a, the imaging unit 12, and the sensor 13 in accordance with a signal from the remote control 10.

In this example, the transmission/reception unit 16 is communicably connected to the transmission/reception unit 35 of a server 40 via a modem 41. Each of the transmission/reception unit 16 and the transmission/reception unit 35 is, for example, a communication module or a communication interface. The method of communication, such as the standard of communication, between the toilet bowl device 20 and the server 40 can be set as appropriate.

The server 40 is an electric circuit including a computer. The server 40 is, for example, a cloud server, and can communicate with the toilet bowl device 20 via the Internet. The server 40 may be a virtual server including a plurality of server groups. The plurality of functional blocks included in the server 40 may be distributed across the plurality of server groups as appropriate.

A controller 36 can communicate with the controller 15 of the toilet bowl device 20 via the transmission/reception unit 35. The controller 36 receives a signal including the detected value detected by the detection unit 11a, the image acquired by the imaging unit 12, and the blood flow information acquired by the sensor 13.

The controller 36 is communicably connected to the female hormone secretion amount fluctuation estimation unit 31, a female hormone secretion amount calculation unit 32, the life stage prediction unit 33, a health state prediction unit 34, the transmission/reception unit 35 (output unit 38), and the memory 37. An electric circuit such as a computer is used as the controller 36. The controller 36 includes, for example, a central processing unit (CPU).

The controller 36 controls the operations of the female hormone secretion amount fluctuation estimation unit 31, the female hormone secretion amount calculation unit 32, the life stage prediction unit 33, the health state prediction unit 34, and the transmission/reception unit 35 (output unit 38).

The memory 37 receives, from the controller 36, and stores (records) the secretion amount (measured value) of the female hormone measured by the measurement unit 11. Further, the memory 37 may receive, from the controller 36, and store the image acquired by the imaging unit 12, the blood flow information acquired by the sensor 13, an estimation result of the female hormone secretion amount fluctuation estimation unit 31, a calculation result of the female hormone secretion amount calculation unit 32, a prediction result of the life stage prediction unit 33, and a prediction result of the health state prediction unit 34. The controller 36 can read data stored in the memory 37 as appropriate. The memory 37 includes, for example, a hard disk drive (HDD) and a solid state drive (SSD). As the memory 37, any desired storage device such as a read-only memory (ROM) or a random access memory (RAM) can be used.

The secretion amount of the female hormone is measured a plurality of times. For example, the secretion amount of the female hormone is measured each time the user uses the toilet bowl device 20. For each of the plurality of measurements, the memory 37 stores the measured secretion amount, the time at which the secretion amount was measured, and information for identifying the user who was measured, in association with each other. That is, the memory 37 accumulates a plurality of pieces of data including the secretion amount of the female hormone and the time of measurement for each user. For each user, the time at which a value of the secretion amount was measured can be referenced from the value of the secretion amount of the female hormone.

The female hormone secretion amount fluctuation estimation unit 31 receives, from the controller 36, the secretion amount (measured value) of the female hormone recorded in the memory 37. The female hormone secretion amount fluctuation estimation unit 31 estimates fluctuation in the secretion amount of the female hormone of the user on the basis of the secretion amounts of the female hormone recorded in the memory 37. That is, the female hormone secretion amount fluctuation estimation unit 31 estimates a temporal change in the secretion amount of the female hormone on the basis of the data including the secretion amount of the female hormone and the time of measurement. More specifically, the female hormone secretion amount fluctuation estimation unit 31 estimates fluctuation in the secretion amount of the female hormone of the user on the basis of past data of a plurality of the secretion amounts accumulated in the memory 37 and present data of the secretion amount.

The estimation result (that is, fluctuation) of the female hormone secretion amount fluctuation estimation unit 31 includes, for example, the secretion amount of the female hormone at a time in the past or in the future. In other words, the female hormone secretion amount fluctuation estimation unit 31 estimates a temporal change in the secretion amount in the past and predicts the secretion amount in the future. The estimation result (that is, fluctuation) of the female hormone secretion amount fluctuation estimation unit 31 includes, for example, a change amount or a speed of change of the secretion amount in a period including at least any of the past, the present, or the future. The female hormone secretion amount fluctuation estimation unit 31 estimates, for example, a cyclical fluctuation in the secretion amount of the female hormone caused by menstruation. The female hormone secretion amount fluctuation estimation unit 31 may estimate a fluctuation cycle of the secretion amount of the female hormone. The female hormone secretion amount fluctuation estimation unit 31 outputs the estimation result to the controller 36.

The method by which the female hormone secretion amount fluctuation estimation unit 31 estimates the fluctuation is not particularly limited. One example is a method of deriving a curve that approximates the fluctuation by fitting a plurality of measured values to an appropriate curve (function).

The female hormone secretion amount calculation unit 32 calculates the amount of the female hormone contained in the bodily fluid or the like on the basis of the detected value of the detection unit 11a. Specifically, the detected value of the detection unit 11a is converted into the secretion amount of the female hormone. In this example, the secretion amount (measured value) of the female hormone measured by the measurement unit 11 is a value calculated by the female hormone secretion amount calculation unit 32 on the basis of the detected value of the detection unit 11a. The female hormone secretion amount calculation unit 32 may be part of the measurement unit 11. The calculation result of the female hormone secretion amount calculation unit 32 is output to the controller 36 and stored in the memory 37.

The life stage prediction unit 33 predicts the time of at least any of menarche, menstruation, or menopause of the user on the basis of the fluctuation in the secretion amount of the female hormone estimated by the female hormone secretion amount fluctuation estimation unit 31. The life stage prediction unit 33 outputs the prediction result to the controller 36.

The controller 36 executes a process of outputting the result predicted by the life stage prediction unit 33. That is, the controller 36 controls the output unit 38 and causes the output unit 38 to output the predicted result. Thus, the output unit 38 outputs the result predicted by the life stage prediction unit 33. In this example, the transmission/reception unit 35 also serves as the output unit 38. The transmission/reception unit 35 is communicably connected to an external device such as a mobile terminal 45, and transmits the prediction result of the life stage prediction unit 33 to the external device. The external device receives the prediction result of the life stage prediction unit 33 and issues a notification of the prediction result.

The notification of the prediction result needs to be issued by at least any of light or sound. Specifically, for example, the prediction result is displayed on a display device. The display device includes any type of display such as a liquid crystal display or an organic electroluminescent (EL) display, or a light-emitting device including a light-emitting diode (LED). The notification of the prediction result may be issued by sound using a speaker or the like of the external device.

For example, in a case where the external device is a smartphone, an application (app) for transmitting and/or receiving data to/from the female life stage prediction system is installed in the smartphone. The notification of the prediction result is displayed on a screen of the smartphone by the app. The external device to which the prediction result of the life stage prediction unit 33 is transmitted may be a terminal owned by the user that has been the measurement target for the secretion amount of the female hormone or a terminal owned by a family member of the user. In other words, the individual that has been the measurement target for the secretion amount or a family member is notified of the prediction result of the life stage prediction unit 33. The person notified of the prediction result may be changed on the basis of whether predicting menarche or menstruation. For example, a family member is notified of the prediction result in the case of menarche, and the individual is notified of the prediction result in the case of menstruation.

The external device is not limited to a mobile terminal such as a smartphone, and may be a computer (personal computer) or another server, or may be provided in a wash stand, the toilet room, or the toilet bowl device. The output unit 38 may output (transmit) the prediction result to an external storage device or a recording medium.

The output by the output unit 38 may be not only external transmission of the prediction result but also notification of the prediction result by the female life stage prediction system 101. In this case, the female life stage prediction system 101 includes, as the output unit 38 separate from the transmission/reception unit 35, a notification unit such as a display device or a speaker for issuing a notification of the prediction result. The notification unit can be provided in the house of the user, such as in the toilet room or the toilet bowl device 20, for example.

Accurate prediction of the time of menarche is known to be difficult. Further, while the time of menstruation can be estimated from the time elapsed from the previous menstruation, accurate prediction of the time of menstruation is difficult in some cases. The secretion amount of the female hormone increases during adolescence and is large in maturation. Then, the secretion amount of the female hormone starts to decrease, which leads to menopause. In due course, the female hormone is no longer secreted, and the woman enters old age. Further, the menstrual cycle and the secretion amount of the female hormone are related to each other. For example, the secretion amount of the female hormone in maturation after menarche changes cyclically with the menstrual cycle. Therefore, the onset of menarche and the time of next menstruation can be predicted by observing fluctuation in the female hormone secretion amount. According to the embodiment, it is possible to estimate, from discharge from the human body, fluctuation in the secretion amount of the female hormone, and to predict the time of at least any of menarche, menstruation, or menopause from the result. Accordingly, it is possible to prepare for menarche or menstruation. Additionally, it is possible to prepare for menopause, which is a period of significant physical change.

The memory 37 stores a female life stage prediction program 100 that causes a computer to execute the female life stage prediction method implemented by the female life stage prediction system 101. For example, the controller 36 reads the female life stage prediction program 100 stored in the memory 37 as needed and processes the female life stage prediction program 100 as needed, causing the computer to execute each process of the female life stage prediction method.

The female life stage prediction program 100 includes a program 100A for executing the processing of each of the female hormone secretion amount fluctuation estimation unit 31, the female hormone secretion amount calculation unit 32, the life stage prediction unit 33, the health state prediction unit 34, and the transmission/reception unit 35. For example, the program 100A causes the controller 36 to execute the processing. For example, the program 100A causes the controller 36 to control the processing of the female hormone secretion amount fluctuation estimation unit 31, the female hormone secretion amount calculation unit 32, the life stage prediction unit 33, the health state prediction unit 34, and the transmission/reception unit 35.

In the example of FIG. 1, the program 100A is stored in the memory 37, and the server 40 is used as at least part of the computer that executes the program according to the embodiment. However, the at least the part of the computer that executes the program is not limited to the server 40 and may be, for example, a smartphone. In other words, at least some of the functions of the server 40 may be provided in the mobile terminal 45. In this case, at least part of the program is, for example, an app installed in the smartphone.

A circuit such as a large-scale-integrated (LSI) circuit or an integrated circuit (IC) may be used for part or all of each block of the female hormone secretion amount fluctuation estimation unit 31, the female hormone secretion amount calculation unit 32, the life stage prediction unit 33, the health state prediction unit 34, and the controller 36. An individual circuit may be used for each block, or a circuit in which some or all of the blocks are integrated may be used. The blocks may be integrally provided, or some of the blocks may be separately provided. Further, in each block, part of the block may be distributed and separately provided. Each of the blocks is not limited to an integrated circuit and may be a dedicated circuit or a general-purpose processor.

In this example, the female hormone secretion amount fluctuation estimation unit 31, the female hormone secretion amount calculation unit 32, the life stage prediction unit 33, the health state prediction unit 34, and the memory 37 are provided in the server 40. However, in the embodiment, at least part of each of the female hormone secretion amount fluctuation estimation unit 31, the female hormone secretion amount calculation unit 32, the life stage prediction unit 33, the health state prediction unit 34, and the memory 37 may be provided in, for example, the toilet bowl device 20, separately from the server 40.

The communication in each case in the embodiment may be any of wireless communication, wired communication, or a combination thereof. The communication may be performed via a network such as the Internet or a local area network (LAN).

FIG. 2(a) and FIG. 2(b) are schematic views for explaining part of the female life stage prediction system according to the embodiment.

FIG. 2(a) is a schematic view illustrating the toilet bowl device 20, and FIG. 2(b) is a schematic view illustrating the sensor 13 measuring the blood flow information.

As illustrated in FIG. 2(a), the toilet bowl device 20 includes the western-style sit-down toilet (hereinafter, simply referred to as a “toilet bowl” for convenience of description) 50 and a toilet seat device 28 provided above the toilet bowl 50. The toilet seat device 28 includes the toilet seat 21, a toilet lid 22, and a main part 23.

The toilet bowl 50 includes the inner toilet bowl 51 (bowl) recessed downward. The toilet bowl 50 receives, in the inner toilet bowl 51, excrement such as urine and feces of the user. The main part 23 of the toilet seat device 28 is provided on the toilet bowl 50 and rearward of the inner toilet bowl 51. The main part 23 pivotally supports the toilet seat 21 and the toilet lid 22 to be openable and closable. As illustrated in FIG. 2(a), in a state in which the toilet lid 22 is open, a user U is seated on a seat face 21f (upper surface) of the toilet seat 21 which is down.

In the example illustrated in FIG. 2(a), the detection unit 11a is installed on the seat face 21f of the toilet seat 21. The detection unit 11a exemplified in FIG. 2(a) detects the female hormone contained in the sweat of the user U or in a volatile component of the sweat. For example, part of the detection unit 11a is installed so as to be exposed to the seat face 21f. The detection unit 11a may come into contact with the back side of the thigh of the user seated on the seat face 21f.

For example, there is a method of measuring the secretion amount of the female hormone by using a pregnancy test kit. However, with such a method, it is not easy to measure fluctuation in the secretion amount. In contrast, according to the embodiment, the secretion amount of the female hormone can be measured simultaneously with the daily act of sitting on the toilet seat 21. For example, the secretion amount of the female hormone can be measured by simply performing the daily act of sitting on the toilet seat 21. Thus, there is no need to prepare a tool such as a pregnancy test kit, and the time and effort required for measurement is reduced. Further, since sweat readily evaporates, it is possible to reduce the need to frequently clean the detection unit 11a and suppress an increase in complexity of the mechanism and failure of the mechanism, for example.

As illustrated in FIG. 2(a), the sensor 13 is provided, for example, on the seat face 21f of the toilet seat 21. With the user U seated on the seat face 21f of the toilet seat 21, the blood flow information can be measured. The sensor 13 is, for example, a laser sensor.

As illustrated in FIG. 2(b), the sensor 13 emits irradiation light (e.g., laser light) toward the back side of the thigh of the user seated on the toilet seat 21 and receives reflected light of the irradiation light. The reflected light includes reflected light from capillaries on the back side of the thigh. Fourier transform or the like can be performed on an output signal of the reflected light on the basis of dynamic light scattering to calculate an index such as the blood flow rate or the pulse wave.

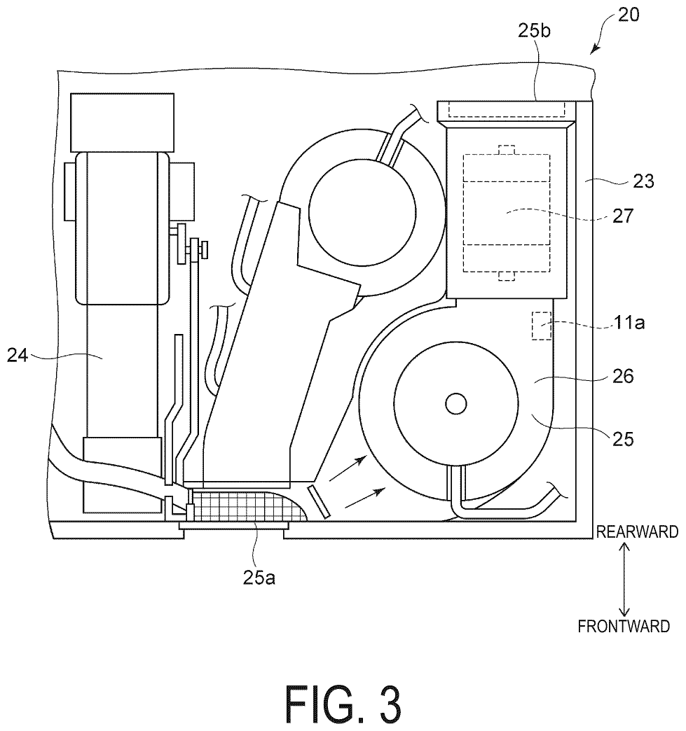

FIG. 3 is a plan view for explaining part of the female life stage prediction system according to the embodiment.

FIG. 3 is a view of the inside of the main part 23 of the toilet bowl device 20 as viewed from above. In the example illustrated in FIG. 3, instead of the detection unit 11a described with reference to FIG. 2(a), the detection unit 11a is provided inside the main part 23.

As illustrated in FIG. 3, a washing nozzle 24 and a duct 25 are provided inside the main part 23. The washing nozzle 24 spouts wash water toward a private part of the user. The duct 25 discharges air present in the inner toilet bowl 51 to the outside of the inner toilet bowl 51.

In the example of FIG. 3, the detection unit 11a is installed in the duct 25. The detection unit 11a detects the female hormone contained in a volatile component of urine of the user. For example, the detection unit 11a is an odor sensor, and the volatile component of urine is urine odor.

The duct 25 is, for example, a duct of a deodorizing device. A blower fan 26 and a deodorizing catalyst 27 are provided inside the duct 25. When the blower fan 26 operates, the air in the inner toilet bowl 51 flows into the duct 25 from an opening 25a provided frontward of the duct 25. The air flowing into the duct 25 is deodorized by coming into contact with the deodorizing catalyst 27, and is discharged to the outside of the toilet bowl device 20 from an opening 25b provided rearward of the duct 25.

The detection unit 11a is positioned on the downstream side of the blower fan 26 and upstream of the deodorizing catalyst 27 in the duct 25. The detection unit 11a comes into contact with the air containing the volatile component of urine of the user and suctioned from the opening 25a, thereby detecting the female hormone contained in the air.

As described above, a sensor that can analyze a component contained in air is disposed in the deodorizing duct of the toilet seat device 28. A blower fan that operates while the user is seated is incorporated in the deodorizing duct. Thus, the air in the inner toilet bowl during urination is suctioned into the deodorizing duct, making it possible to expose the sensor to the air.

According to this embodiment, the secretion amount of the female hormone can be measured simultaneously with the daily act of sitting on the toilet seat 21 and excreting. For example, the secretion amount of the female hormone can be measured simply by performing the daily act of excretion. Thus, there is no need to prepare a tool such as a pregnancy test kit, and the time and effort required for measurement is reduced. Further, for example, since the volatile component (urine odor) of the urine is measured, it is possible to reduce the need to frequently clean the detection unit 11a and suppress an increase in complexity of the mechanism and failure of the mechanism.

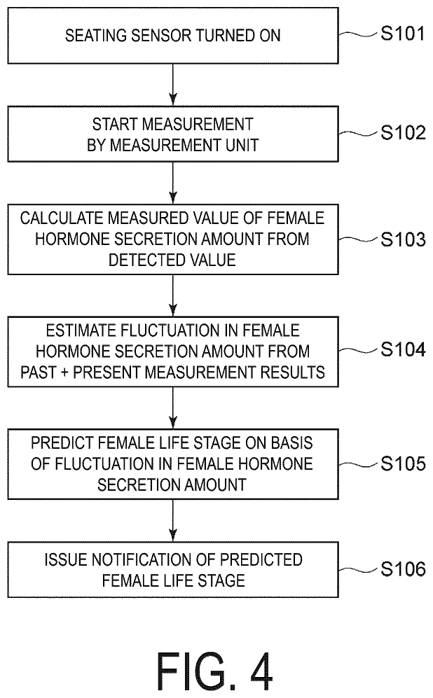

FIG. 4 is a flowchart illustrating an example of the female life stage prediction method of the female life stage prediction system according to the embodiment.

A computer (such as the controller 36) of the female life stage prediction system executes each process of the female life stage prediction method described below on the basis of the female life stage prediction program 100, for example. However, some or all of the processes of the female life stage prediction method according to the embodiment may be executed on the basis of operations of hardware that does not require a program.

When the seating sensor 14 detects that the user is seated (step S101), measurement by the measurement unit 11 is started (S102). Specifically, upon receipt, from the seating sensor 14, of a signal indicating that the user is seated, the controller 15 controls the detection unit 11a to detect the female hormone.

The detected value of the female hormone detected by the detection unit 11a is transmitted from the controller 15 to the female hormone secretion amount calculation unit 32 via the controller 36 on the server 40 side. The female hormone secretion amount calculation unit 32 calculates the secretion amount (measured value) of the female hormone from the detected value (step S103). The secretion amount of the female hormone calculated in step S103 is stored in the memory 37.

The female hormone secretion amount fluctuation estimation unit 31 estimates the fluctuation in the secretion amount of the female hormone of the user on the basis of past data of the secretion amount accumulated in the memory 37 and present data of the secretion amount (step S104).

The life stage prediction unit 33 predicts the female life stage on the basis of the fluctuation in the secretion amount of the female hormone estimated by the female hormone secretion amount fluctuation estimation unit 31 (step S105).

The output unit 38 outputs the prediction result of the life stage prediction unit 33. Specifically, the transmission/reception unit 35 transmits the prediction result of the life stage prediction unit 33 to the mobile terminal 45. Accordingly, the mobile terminal 45 issues a notification of the predicted female life stage (step S106).

As described above, the measurement unit 11 starts measurement when the seating sensor 14 detects that the user is seated on the toilet seat 21. Thus, the female hormone secretion amount can be automatically measured by performing the daily act of sitting on a toilet seat. Since the secretion amount can be recorded every day without forgetting, accuracy of estimation of the fluctuation in the secretion amount and accuracy of life stage prediction based on the estimated fluctuation can be improved, for example. For example, when the seating of the user is detected in step S101, the subsequent steps S102 to S106 are automatically sequentially executed.

The female life stage prediction system 101 performs individual authentication of the user before executing the female life stage prediction method. For example, information for identifying the user (identification (ID) information) is registered in advance in the female life stage prediction system 101. The controller 15 or the controller 36 of the female life stage prediction system 101 identifies the user by confirming the corresponding registered ID information of the user. Such individual authentication may be automatically performed by detecting a load applied to the toilet seat 21 by the seated user, or may be performed by the user inputting ID information from the remote control 10 or the like, for example. In a case where the user is authenticated as a female, the female life stage prediction system 101 can execute the female life stage prediction method.

For example, the imaging unit 12 captures an image of the inside of the inner toilet bowl 51 simultaneously with measurement of the secretion amount of the female hormone. Further, the sensor 13 acquires the blood flow information of the user simultaneously with measurement of the secretion amount of the female hormone. Note that the term “simultaneously” need not be “fully simultaneously” and needs to be, for example, during one seating action of the user.

The detection of the female hormone by the detection unit 11a, the imaging by the imaging unit 12, and the measurement by the sensor 13 may be performed from the start to the end of the excretion action of the user, for example. The detection of the female hormone by the detection unit 11a, the imaging by the imaging unit 12, and the measurement by the sensor 13 may be continuously or intermittently implemented from detection of seating to detection of unseating of the user by the seating sensor 14, for example.

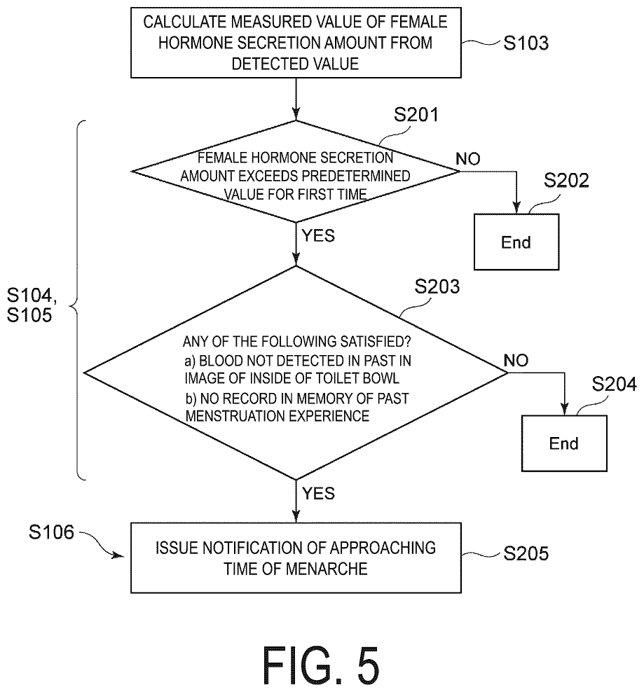

FIG. 5 is a flowchart illustrating an example of the female life stage prediction method of the female life stage prediction system according to the embodiment.

FIG. 5 illustrates an example of steps S103 to S106 described with reference to FIG. 4. In the example of FIG. 5, step S201 to step S204 are executed as step S104 and step S105, and step S205 is executed as step S106.

The female hormone secretion amount fluctuation estimation unit 31 estimates the fluctuation in the secretion amount of the female hormone of the user. The life stage prediction unit 33 determines whether the secretion amount of the female hormone at a future time estimated by the female hormone secretion amount fluctuation estimation unit 31 exceeds a predetermined value for the first time (step S201). When the secretion amount of the female hormone at the future time estimated by the female hormone secretion amount fluctuation estimation unit 31 exceeds the predetermined value (step S201: YES), step S203 is executed. When the secretion amount of the female hormone at the future time estimated by the female hormone secretion amount fluctuation estimation unit 31 does not exceed the predetermined value (step S201: NO), the processing ends (step S202).

If either condition a) or condition b) is satisfied (step S203: YES), step S205 is executed. In step S205, the output unit 38 outputs the prediction result of the life stage prediction unit 33, that is, information indicating that the time of menarche is approaching. Accordingly, a notification indicating that the time of menarche is approaching is issued. When neither condition a) nor condition b) is satisfied (step S203: NO), the processing ends (step S204).

Condition a) is that blood (menstrual blood) is not identified in past images captured by the imaging unit 12. Condition b) is that there is no record in the memory 37 that the current user experienced menstruation (menarche) in the past. Note that condition b) is not met in a case where the user manually inputs information into the system that she experienced menstruation (menarche) in the past.

As described above, the life stage prediction unit 33 predicts the onset of menarche when the estimated fluctuation in the secretion amount of the female hormone satisfies a predetermined condition. According to the embodiment, the onset of menarche can be predicted, and thus the user can prepare. For example, sanitary products can be prepared before the user's first menstruation and advice can be given in advance on how to deal with menstruation, including how to use the sanitary products. As a result, menarche can be experienced with less worry.

In the example described above, the predetermined condition for predicting the onset of menarche is that the estimated value of the secretion amount of the female hormone at a future time exceeds a predetermined value. The predetermined condition for predicting the onset of menarche may be that the increase amount, the speed of increase, the change rate of the speed of increase, or the like of the estimated secretion amount of the female hormone exceeds a predetermined value. The predetermined condition is not limited to that described above and need only be determined as appropriate to facilitate prediction of the onset of menarche. For example, the predetermined condition can be determined on the basis of statistical data of a correlation between the secretion amount of the female hormone and menarche (time of menarche). Note that the statistical data is, for example, data acquired in advance for a plurality of users, and needs to be data representing a case of a typical woman. The same applies to statistical data described below.

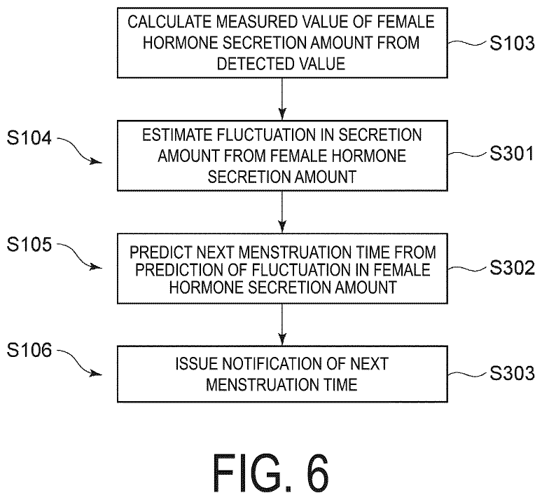

FIG. 6 is a flowchart illustrating an example of the female life stage prediction method of the female life stage prediction system according to the embodiment.

FIG. 6 illustrates an example of steps S103 to S106 described with reference to FIG. 4. In the example of FIG. 6, step S301 is executed as step S104, step S302 is executed as step S105, and step S303 is executed as step S106.

The female hormone secretion fluctuation estimation unit 31 estimates the fluctuation in the secretion amount of the female hormone of the user (step S301). The life stage prediction unit 33 predicts the time of next menstruation from the estimated fluctuation in the secretion amount of the female hormone (step S302). The output unit 38 outputs the prediction result of the life stage prediction unit 33. Accordingly, a notification of the predicted time of next menstruation is issued (step S303).

Thus, after the user experiences menarche, the life stage prediction unit 33 predicts the time of next menstruation on the basis of the cyclical fluctuation in the female hormone estimated by the female hormone secretion amount fluctuation estimation unit 31. Since the fluctuation in the secretion of the female hormone changes so as to cyclically fluctuate after menarche, it is possible to predict the time of menstruation, prepare sanitary products, and prepare for health problems.

For example, in the cyclical change in the secretion amount of the female hormone, the time at which the estimated fluctuation in the female hormone (e.g., the secretion amount, the change amount of the secretion amount, or the speed of change) satisfies a predetermined condition can be predicted as the time of menstruation. For example, of times at which the estimated future secretion amount, change amount, or speed of change of the female hormone exceeds a predetermined value, the closest time can be predicted as the time of next menstruation.

The predetermined condition for predicting the time of menstruation need only be determined as appropriate to facilitate prediction of the time of menstruation. For example, the predetermined condition may be determined on the basis of statistical data of a correlation between the secretion amount of the female hormone and the menstrual cycle (time of menstruation), or may be determined on the basis of a correlation between past secretion amounts of the female hormone and the menstrual cycle of the user.

Note that, at the first menstruation after menarche, the cyclical fluctuation in the female hormone secretion amount occurs for the first time, and thus the fluctuation of the individual is not known. Therefore, for the first menstruation after menarche, the time may be predicted on the basis of a typical menstrual cycle (28 days) or by using a model value obtained by calculating an average time from menarche to the next menstruation using data of other users of the device. For the third and subsequent menstruation, future menstruation may be predicted on the basis of a past history of the individual. Alternatively, since past records are unavailable for several times, the time may be predicted by the same method as that for the second menstruation.

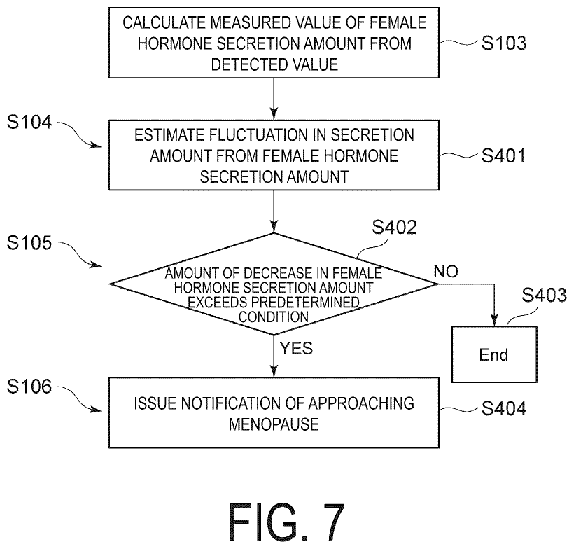

FIG. 7 is a flowchart illustrating an example of the female life stage prediction method of the female life stage prediction system according to the embodiment.

FIG. 7 illustrates an example of steps S103 to S106 described with reference to FIG. 4. In the example of FIG. 7, step S401 is executed as step S104, steps S402 to S403 are executed as step S105, and step S404 is executed as step S106.

The female hormone secretion amount fluctuation estimation unit 31 estimates the fluctuation in the secretion amount of the female hormone of the user (step S401). The life stage prediction unit 33 determines whether, in the estimated fluctuation in the secretion amount of the female hormone, the decrease amount of the secretion amount of the female hormone exceeds a predetermined condition (predetermined value). When the decrease amount of the secretion amount of the female hormone exceeds the predetermined value (step S402: YES), step S404 is executed. In step S404, the output unit 38 outputs the prediction result of the life stage prediction unit 33, that is, the prediction result indicating that menopause is approaching. Accordingly, a notification indicating that menopause is approaching is issued. When the decrease amount of the secretion amount of the female hormone does not exceed the predetermined value (step S402: NO), the processing ends (step S403).

Thus, the life stage prediction unit 33 predicts the approach of menopause or the possibility of the onset of menopausal symptoms on the basis of a decreasing trend of the secretion amount of the female hormone. This can be useful for health care management, for example.

For example, the life stage prediction unit 33 predicts that menopause is approaching in a case where the decreasing trend of the secretion amount of the female hormone significantly changes in the estimated future fluctuation in the secretion amount of the female hormone (or in the measured value of the secretion amount of the female hormone). For example, in a case where the decreasing trend of the secretion amount of the female hormone significantly changes, the life stage prediction unit 33 considers the user to have reached menopause and predicts the possibility of the occurrence of menopausal symptoms.

More specifically, for example, the life stage prediction unit 33 predicts that menopause is approaching in a case where the speed of decrease in the secretion amount of the female hormone is greater than a predetermined value in the estimated future fluctuation in the secretion amount of the female hormone. Further, for example, the life stage prediction unit 33 predicts that there is a possibility of the occurrence of menopausal symptoms in a case where the speed of decrease in the secretion amount of the female hormone in the current fluctuation in the secretion amount of the female hormone is greater than a predetermined value.

Note that the speed of decrease is, for example, a speed at which the average value (moving average) of the secretion amount in the most recent predetermined period (e.g., a period of one or more cycles of menstruation) decreases. In other words, the speed of decrease is the decrease speed of the secretion amount excluding decreases due to the cyclical fluctuation associated with menstruation.

The predetermined condition for predicting the approach of menopause or the possibility of the occurrence of menopausal symptoms needs to be determined as appropriate to facilitate prediction of the approach of menopause or the possibility of the occurrence of menopausal symptoms. For example, the predetermined condition may be determined on the basis of statistical data of a correlation between the secretion amount of the female hormone and menopause.

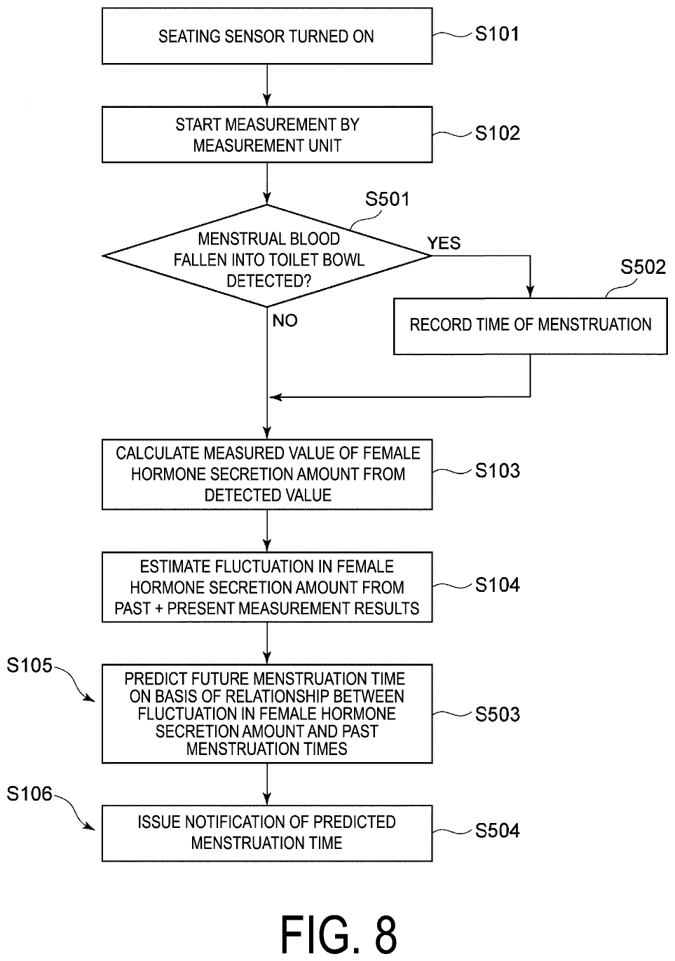

FIG. 8 is a flowchart illustrating an example of the female life stage prediction method of the female life stage prediction system according to the embodiment.

In the example illustrated in FIG. 8, the female life stage prediction system 101 executes step S501 and step S502 in addition to steps S101 to S106 described with reference to FIG. 4. Further, in the example of FIG. 8, step S503 is executed as step S105, and step S504 is executed as step S106.

After step S102, the imaging unit 12 acquires an image of the inside of the inner toilet bowl simultaneously with the measurement of the secretion amount of the female hormone. The acquired image is transmitted from the controller 15 to the controller 36 on the server 40 side. For example, the controller 36 determines whether menstrual blood is identified in the image (step S501).

In a case where menstrual blood that has fallen in the toilet bowl is detected in the image (step S501: YES), it is determined that the present time is the time of actual menstruation of the user. The memory 37 stores the time at which the image of the detected menstrual blood was captured as the time of actual menstruation (step S502). In a case where menstrual blood that has fallen in the toilet bowl is not detected in the image (step S501: NO), it is determined that the present time is not the time of actual menstruation of the user. Note that, as the menstrual blood detection method, a known image recognition method needs to be used as appropriate. The image may include a moving image.

In step S103, the measured value of the secretion amount of the female hormone is calculated. In step S104, the fluctuation in the secretion amount of the female hormone is estimated.

The life stage prediction unit 33 predicts the time of future menstruation on the basis of the relationship between the estimated fluctuation in the secretion amount of the female hormone and the times of past actual menstruation stored in the memory 37 (step S503).

The output unit 38 outputs the prediction result of the life stage prediction unit 33. Accordingly, a notification of the predicted time of menstruation is issued (step S504).

The typical cycle of menstruation is 25 to 38 days. The cycle varies from person to person. The cycle also varies by age and other factors (e.g., high stress), even for the same person. Further, the female hormone secretion amount at the time when menstruation begins is not a constant value and varies by person, age, and living environment. Furthermore, the menstrual period is three to seven days and varies by person, age, and the like. Therefore, in a case where the time of next menstruation is predicted on the basis of the female hormone secretion amount or the cyclical fluctuation thereof alone, there may be room to improve the accuracy of the prediction result.

Therefore, if the female hormone secretion amount of a female who is actually menstruating is known, it is possible to associate the fluctuation in the female hormone secretion amount and the time of menstruation of the individual with each other. For example, the menstruation start time and the menstruation end time are recorded in association with the fluctuation (curve) of past female hormone secretion amounts estimated on the basis of daily measurement. Thus, of several days on which the start of menstruation is predicted in the future fluctuation curve of the female hormone secretion amount, the day on which the female hormone secretion amount reaches that in the actual menstruation start time in the past is estimated as the next menstruation start time. With repetition of this, the accuracy of prediction is further improved. Further, even if the relationship between the female hormone secretion amount and the time of menstruation changes as the user ages, it is possible to also change the prediction of the time of menstruation accordingly.

Thus, the life stage prediction unit 33 predicts the time of subsequent menstruation on the basis of the time of actual menstruation determined on the basis of the image captured by the imaging unit 12 and the estimated cyclical fluctuation of the female hormone. By confirmation of the time of actual menstruation based on the image captured by the imaging unit 12, prediction accuracy of the time of subsequent menstruation can be improved. For example, the life stage prediction unit 33 can correct the prediction of the menstrual cycle on the basis of the time of actual menstruation determined on the basis of the image.

For example, on the basis of the correlation between the time of actual menstruation determined on the basis of the image and the measured value of the secretion amount of the female hormone, the life stage prediction unit 33 obtains a feature of the fluctuation in the secretion amount of the female hormone at the time of actual menstruation (e.g., secretion amount and speed of change thereof). The feature may be obtained on the basis of a correlation between the time of actual menstruation and the fluctuation in the secretion amount estimated from the measured value.

The life stage prediction unit 33 changes the predetermined condition for predicting the time of menstruation on the basis of the obtained feature. For example, as the time of menstruation, the life stage prediction unit 33 can predict a time at which the estimated fluctuation in the secretion amount of the female hormone matches the feature of the secretion amount at the time of actual menstruation.

The controller 36 may suggest an action to the user on the basis of the predicted time of menstruation and calendar information. As a result, the prediction result of the time of menstruation can be useful in planning. For example, the prediction result of the time of menstruation can be used to plan vacation days and schedule travel and other activities.

The calendar information is stored in the memory 37. The calendar information includes, for example, the user's work schedule and the user's day off schedule. For example, the controller 36 refers to the calendar information of the predicted time of menstruation and generates action suggestion information. Specifically, in a case where the predicted time of menstruation is a weekend, the controller 36 generates action suggestion information that encourages the user to plan to take a rest on that weekend. Alternatively, the controller 36 generates action suggestion information that encourages the user to plan a trip or the like on days off other the predicted time of menstruation. The generated action suggestion information is output from the output unit 38. As a result, the user is notified of action suggestions corresponding to the action suggestion information.

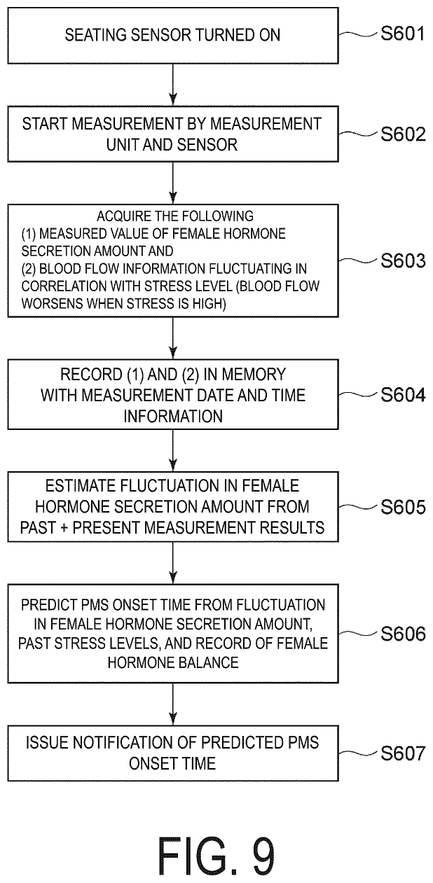

FIG. 9 is a flowchart illustrating an example of the female life stage prediction method of the female life stage prediction system according to the embodiment.

When the seating sensor 14 detects that the user is seated (step S601), measurement by the measurement unit 11 and the sensor 13 is started (step S602). For example, when receiving from the seating sensor 14 a signal indicating that the user is seated, the controller 15 controls the sensor 13 to start measurement of the blood flow information.

The female hormone secretion amount calculation unit 32 calculates the secretion amount (measured value) of the female hormone. The sensor 13 acquires the measured value of the blood flow information (step S603). Here, the blood flow information acquired by the sensor 13 is a value that fluctuates in correlation with the stress level of the user. As an example, the sensor 13 measures the blood flow rate. For example, when the stress of the user is high, the blood flow rate may decrease.

The measurement result of the blood flow information by the sensor 13 is transmitted from the controller 15 to the health state prediction unit 34 via the controller 36 on the server 40 side. For example, the health state prediction unit 34 calculates the stress level of the user from the blood flow information measured in step S603.

For example, the stress level is an index value indicating the magnitude of physical or mental stress of the user. As an example, the stress level is an index value that represents a classified magnitude of the value of the blood flow information. Although depending on the type of the acquired blood flow information, the health state prediction unit 34 determines that the stress level is low in a case where the value of the blood flow information is high, for example. Depending on the type of the acquired blood flow information, the health state prediction unit 34 may determine that the stress level is high in a case where the value of the blood flow information is high. The stress level needs to be an index correlated with the value of the blood flow information.

The measured value of the secretion amount of the female hormone, the blood flow information acquired by the sensor 13, the stress level calculated by the health state prediction unit 34 on the basis of the blood flow information, and the time at which the secretion amount and the blood flow information are measured are stored in the memory 37 (step S604). That is, the stress level is stored in association with the secretion amount of the female hormone measured when the blood flow information is acquired. The stress level when the value of the secretion amount is measured can be referenced from the value of the secretion amount of the female hormone.

In this way, each time the secretion amount of the female hormone is measured, the blood flow information at that time is measured, and the stress level at that time is calculated. Each time the secretion amount of the female hormone is measured, the stress level at that time is stored in the memory 37 in association with the measured secretion amount of the female hormone.

The female hormone secretion amount fluctuation estimation unit 31 estimates the fluctuation in the secretion amount of the female hormone of the user on the basis of past data of the secretion amounts accumulated in the memory 37 and present data of the secretion amount (step S605).

The health state prediction unit 34 predicts, for example, the onset time of premenstrual syndrome (PMS) on the basis of the fluctuation in the secretion amount of the female hormone estimated by the female hormone secretion amount fluctuation estimation unit 31 and the relationship between the past stress levels and the past secretion amounts of the female hormone recorded in the memory 37 (step S606). The output unit 38 outputs the prediction result of the health state prediction unit 34. Accordingly, a notification of the predicted onset time of PMS is issued (step S607).

Three to ten days before menstruation, mental and physical health problems may occur due to the effects of fluctuations in female hormones. This symptom is referred to as premenstrual syndrome (PMS). If PMS can be predicted in advance, PMS symptoms can be suppressed to a low level by taking measures in advance, which can achieve a comfortable way of life even before menstruation.

When PMS develops, the stress level becomes high due to mental and physical distress.

When the autonomic nervous system becomes unbalanced due to stress, blood vessels contract and blood flow worsens. Therefore, the stress level can be identified by measuring the blood flow state. The blood flow state can be measured by using, for example, an optical sensor embedded in the seat face of the toilet seat.

As long as the relationship between the stress level and the female hormone secretion amount is recorded in association with each other, the female hormone secretion amount corresponding to a high stress level can be identified for each individual. Then, from the prediction result of the fluctuation in the female hormone secretion amount, it is possible to predict the onset time of PMS symptoms that cause high stress levels.

Specifically, the health state prediction unit 34 can predict future fluctuation in the stress level from the estimated fluctuation in the secretion amount of the female hormone on the basis of the correlation between past stress levels and past secretion amounts of the female hormone. As a result, the health state prediction unit 34 predicts the onset of a rise in the stress level. For example, the health state prediction unit 34 predicts that the time at which the stress level rises is near, or predicts at least any of the start or the end of the time period in which the stress level is high. For example, the health state prediction unit 34 predicts the onset of a rise in the stress level before menstruation. The rise in the stress level before menstruation may correspond to premenstrual syndrome.

For example, on the basis of the correlation between past stress levels and past secretion amounts of the female hormone, a feature of the fluctuation in the secretion amount of the female hormone (e.g., secretion amount or speed of change thereof) at the time of an increase in the stress level is known. A condition for predicting a rise in the stress level can be defined on the basis of this feature. For example, the health state prediction unit 34 predicts the time at which the estimated fluctuation in the secretion amount of the female hormone (e.g., secretion amount, change amount of secretion amount, or speed of change) satisfies the condition, as the time at which the stress level increases. For example, the health state prediction unit 34 can predict the time at which the secretion amount, the change amount, or the speed of change of the female hormone exceeds a predetermined value as the time at which the stress level increases. For example, the time at which the estimated fluctuation in the secretion amount of the female hormone matches the feature of the secretion amount of the female hormone when the stress level increased in the past can be predicted as the time at which the stress level increases.

The estimation of the fluctuation in the secretion amount of the female hormone is based on, for example, the present measurement result of the most recent secretion amount. The present value of the most recent stress level may be reflected in the prediction of the fluctuation in stress level. For example, the stress level predicted from the fluctuation in the secretion amount of the female hormone may be adjusted according to the present magnitude of the stress level.

The condition for predicting a rise in the stress level need only be determined as appropriate to facilitate prediction of a rise in the stress level (e.g., health problems such as the onset of premenstrual syndrome). For example, the condition for predicting a rise in the stress level may be determined on the basis of statistical data of a correlation between the secretion amount of the female hormone and the stress level, or on the basis of the correlation between the secretion amounts of the female hormone and the stress levels of the user in the past.

As described above, the health state prediction unit 34 predicts the onset of health problems caused by a rise in the stress level of the user before menstruation on the basis of the relationship between the past stress levels stored in the memory 37 and the secretion amounts of the female hormone associated with the stress levels, and the present secretion amount of the female hormone of the user newly measured. On the basis of the relationship between the female hormone and the stress levels in the past, it is possible to predict health problems (e.g., premenstrual syndrome) of the user before menstruation from the most recent measurement result.

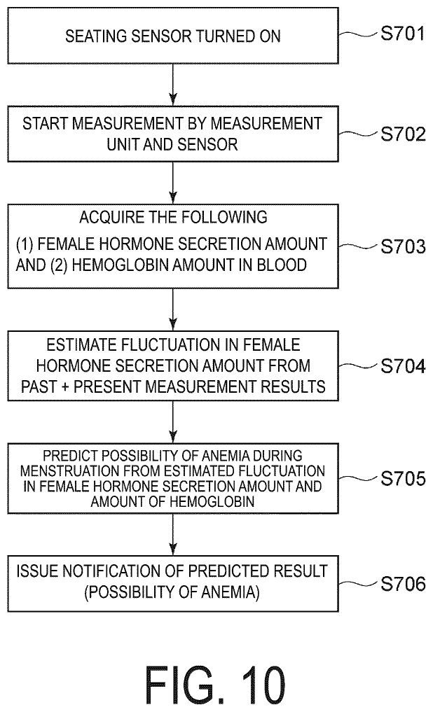

FIG. 10 is a flowchart illustrating an example of the female life stage prediction method of the female life stage prediction system according to the embodiment.

When the seating sensor 14 detects that the user is seated (step S701), measurement by the measurement unit 11 and the sensor 13 is started (step S702).

The female hormone secretion amount calculation unit 32 calculates the secretion amount (measured value) of the female hormone. The sensor 13 acquires the measured value of the blood flow information (step S703). In this example, the blood flow information acquired by the sensor 13 is the hemoglobin amount in the blood of the user. The blood flow information may be an estimated value of the hemoglobin concentration in the blood. For example, each time the secretion amount of the female hormone is measured, the hemoglobin amount at that time is measured and transmitted to the health state prediction unit 34.

The female hormone secretion amount fluctuation estimation unit 31 estimates the fluctuation in the secretion amount of the female hormone of the user on the basis of past data of the secretion amounts accumulated in the memory 37 and present data of the secretion amount (step S704).

The health state prediction unit 34 predicts the possibility of anemia of the user on the basis of the fluctuation in the secretion amount of the female hormone estimated by the female hormone secretion amount fluctuation estimation unit 31 and the blood flow information measured by the sensor 13 (step S705). The output unit 38 outputs the prediction result of the health state prediction unit 34. Accordingly, a notification of the predicted possibility of anemia is issued (step S706).

Since menstruation involves bleeding, iron deficiency may occur. Iron deficiency leads to insufficient production of hemoglobin, resulting in anemia. However, a decrease in hemoglobin does not always result in anemia. Nor is anemia always present during menstruation. However, in a case where hemoglobin decreases during menstruation, the decrease in hemoglobin can be regarded as due to iron deficiency, and thus it is possible to predict the possibility of anemia due to iron deficiency.

Here, the female hormone secretion amount is measured while the amount of hemoglobin is measured with a sensor that can measure blood flow. With the female hormone secretion amount being suppressed during menstruation, the possibility of anemia is predicted in a case where there is a decrease in the hemoglobin amount during menstruation. This makes it possible to suppress anemia or suppress the symptoms of anemia through advance measures (medicine, supplementation, diet).

Specifically, in step S705, the health state prediction unit 34 predicts that there is a possibility of anemia in a case where the present blood flow information (hemoglobin amount) is lower than a reference value and the near future secretion amount of the female hormone in the estimated fluctuation in the secretion amount of the female hormone is lower than a predetermined value. Alternatively, the health state prediction unit 34 may predict that there is a possibility of anemia in a case where the present blood flow information is lower than the reference value and the present measured value of the secretion amount of the female hormone (or the present secretion amount of the female hormone in the estimated fluctuation in the secretion amount of the female hormone) is lower than the predetermined value.

As the reference value of the hemoglobin amount for predicting anemia, a numerical value of a typical diagnostic criterion can be used, for example. The predetermined value of the secretion amount of the female hormone for predicting anemia need only be determined as appropriate to facilitate prediction of anemia. For example, the predetermined value may be determined on the basis of statistical data of a correlation between the occurrence of anemia and the secretion amount of the female hormone, or on the basis of a relationship between the occurrence of anemia and the secretion amounts of the female hormone of the user in the past.

As described above, the health state prediction unit 34 predicts the possibility of anemia of the user on the basis of the blood flow information and the estimated fluctuation in the secretion amount of the female hormone. By taking into account both the blood flow information and the secretion amount of the female hormone, it is possible to predict the possibility of anemia with high accuracy.

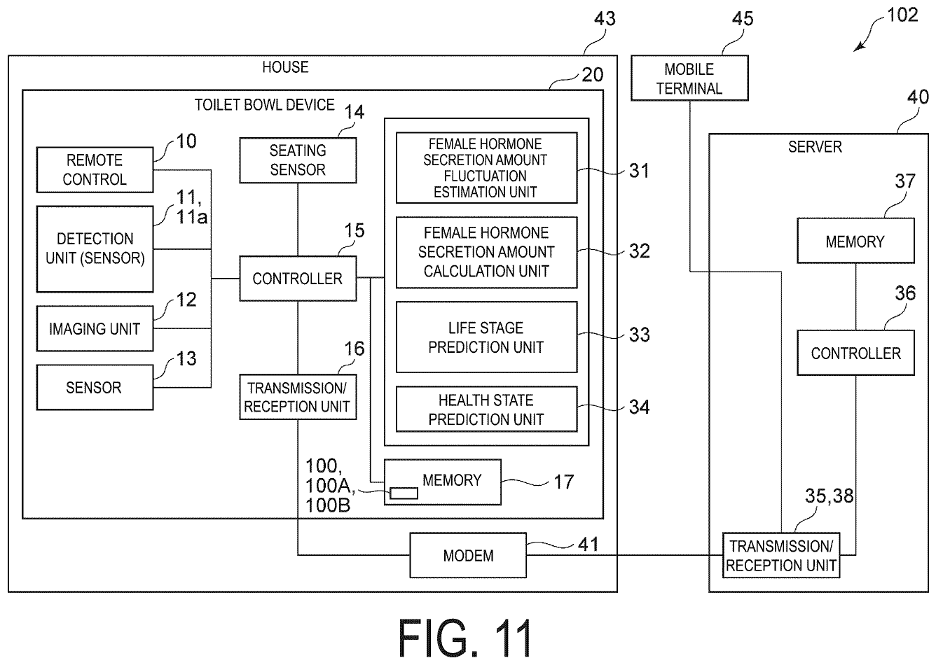

FIG. 11 is a schematic block diagram illustrating another female life stage prediction system according to the embodiment.

FIG. 11 illustrates a female life stage prediction system 102 that executes the female life stage prediction method according to the embodiment.

In this example, the female hormone secretion amount fluctuation estimation unit 31, the female hormone secretion amount calculation unit 32, the life stage prediction unit 33, and the health state prediction unit 34 are provided in the toilet bowl device 20. The controller 15 can communicate with the female hormone secretion amount fluctuation estimation unit 31, the female hormone secretion amount calculation unit 32, the life stage prediction unit 33, and the health state prediction unit 34. The controller 15 controls the female hormone secretion amount fluctuation estimation unit 31, the female hormone secretion amount calculation unit 32, the life stage prediction unit 33, and the health state prediction unit 34.

In this example, the toilet bowl device 20 includes a memory 17 (recording unit) that can communicate with the controller 15. As the memory 17, any storage device such as a ROM or a RAM can be used.

The memory 17 stores the secretion amount (measured value) of the female hormone measured by the measurement unit 11. The memory 17 may store the image acquired by the imaging unit 12, the blood flow information acquired by the sensor 13, the estimation result of the female hormone secretion amount fluctuation estimation unit 31, the calculation result of the female hormone secretion amount calculation unit 32, the prediction result of the life stage prediction unit 33, and the prediction result of the health state prediction unit 34.

Note that, in this example, the female life stage prediction program 100 is stored not in the memory 37 but in the memory 17. The controller 15 reads the female life stage prediction program 100 stored in the memory 17 as needed and processes the female life stage prediction program 100 as needed, thereby causing the computer to execute each process of the female life stage prediction method.

The female life stage prediction program 100 may include a program 100B that controls operations of the detection unit 11a, the imaging unit 12, and the sensor 13. For example, the program 100B causes the controller 15 to execute processing. For example, the program 100B causes the controller 15 to control the processing in the detection unit 11a, the imaging unit 12, and the sensor 13.

Note that the female life stage prediction program 100 may be distributed and stored in a plurality of recording media, or may cause a plurality of controllers to execute the female life stage prediction method. That is, for example, part of the female life stage prediction program 100 may be stored in the memory 37 and executed by the controller 36, and another part of the female life stage prediction program 100 may be stored in the memory 17 and executed by the controller 15.

A fluctuation state of the secretion amount of the female hormone of the user is transmitted to the controller 36 of the server 40. The fluctuation state of the secretion amount of the female hormone is, for example, measured values of the secretion amount of the female hormone or the fluctuation in the secretion amount of the female hormone estimated by the female hormone secretion amount fluctuation estimation unit 31. The transmitted measured values are a plurality of pieces of data in at least any of the past or the present.

The controller 36 of the server 40 compares the transmitted fluctuation state with a female hormone secretion amount fluctuation model. The output unit 38 outputs menstrual state information of the user based on the result of the comparison.

The female hormone secretion amount fluctuation model is a model representing a temporal change in the secretion amount of the female hormone in a typical user. That is, the female hormone secretion amount fluctuation model is data representing average fluctuation in the secretion amount of the female hormone. For example, the female hormone secretion amount fluctuation model is statistical data of temporal changes in the secretion amount of the female hormone. The female hormone secretion amount fluctuation model is, for example, the average value of fluctuations in the secretion amounts of the female hormone of a plurality of users.

For example, the controller 36 compares the secretion amount of the female hormone of the user with the secretion amount of the female hormone in the female hormone secretion amount fluctuation model. Alternatively, the controller 36 may compare the change amount (or speed of change) of the secretion amount of the female hormone of the user with the change amount (or speed of change) of the secretion amount of the female hormone in the female hormone secretion amount fluctuation model. Alternatively, the controller 36 may compare the fluctuation cycle of the secretion amount of the female hormone of the user with the fluctuation cycle of the secretion amount of the female hormone in the female hormone secretion amount fluctuation model.

The menstrual state information of the user is information including the result of comparison described above. That is, the menstrual state information of the user includes, for example, information indicating whether the secretion amount (or change amount of the secretion amount) of the female hormone of the user is large or small compared with that of the female hormone secretion amount fluctuation model. Alternatively, the menstrual state information of the user includes, for example, information indicating whether the fluctuation cycle of the secretion amount of the female hormone of the user is long or short compared with that of the female hormone secretion amount fluctuation model.

The menstrual state information of the user may include information obtained by determining a difference between the fluctuation state of the secretion amount of the female hormone of the user and that of the female hormone secretion amount fluctuation model. Specifically, the menstrual state information of the user may include information obtained by determining a difference between the secretion amount (or fluctuation cycle) of the female hormone of the user and the secretion amount (or fluctuation cycle) in the female hormone secretion amount fluctuation model. For example, the heaviness of menstruation may be determined on the basis of the magnitude of the difference.

In this way, the user can grasp the results of the comparison between her own fluctuation in the secretion amount of the female hormone and that of the typical female hormone secretion amount fluctuation model. For example, the user can grasp a difference such as whether her own cycle of menstruation is long or short compared with a typical cycle. This makes it possible to, for example, provide advice, such as seeking consultation with a doctor, and reference information to the user.

The processing in the methods illustrated in the foregoing embodiments can be executed on the basis of a program that is software. A general-purpose computer system can store the program in advance and read the program so that effects similar to the effects obtained by the methods of the embodiments described above can be obtained.

The program according to the embodiments is not limited to the form of a program for causing a computer to execute the methods described above (a form in which the program is provided in a computer), and may be in the form of a computer-readable recording medium. As the recording medium, for example, a CD-ROM (-R/-RW), a magneto-optical disk, a hard disk (HD), a DVD-ROM (-R/-RW/-RAM), a flexible disk (FD), a flash memory, a recording medium similar to these, and other various ROMS, RAMS, and the like can be used. The storage format may be any format as long as the recording medium is readable by a computer or an embedded system. When the computer reads the program from this recording medium and causes the central processing unit (CPU) to execute the instructions described in the program on the basis of the program, operations similar to those of the embodiments described above can be realized. Of course, in a case where the computer acquires or reads the program, the program may be acquired or read through a network.

Further, an operating system (OS) operating on a computer, database management software, middleware (MW) operating on a network, or the like may execute part of each process for realizing the embodiments on the basis of instructions of the program installed from a recording medium onto a computer or an embedded system.

Furthermore, the recording medium in the embodiments is not limited to a recording medium independent of a computer or an embedded system, and includes a recording medium in which the program transmitted via a local area network (LAN), the Internet, or the like is downloaded and stored or temporarily stored. Further, the number of recording media is not limited to one, and a case where the processing in the embodiments is executed from a plurality of recording media is also included in the form of the recording media in the embodiments. The recording medium may have any configuration.

Note that the computer or the embedded system in the embodiments is for executing each process in the embodiments on the basis of the program stored in the recording medium, and may be configured as any of a device composed of one of a personal computer, a microcomputer, or the like, or a system in which a plurality of devices are connected to each other via a network.

Further, the computer in the embodiments is not limited to a personal computer, and also includes an arithmetic processing device included in an information processing device, a microcomputer, and the like, and collectively refers to devices and apparatuses that can realize the functions in the embodiments by the program.

In the embodiments described above, the life stage prediction unit 33 may predict the life stage on the basis of the secretion amount of one type of female hormone, or may predict the life stage on the basis of a combination of the secretion amounts of two or more types of female hormones. Further, gonadotropin (luteinizing hormone (LH) and follicle-stimulating hormone (FSH)) that controls the production and secretion of female hormones may be used instead of female hormones. Alternatively, urinary metabolites (estradiol glucuronic conjugate, pregnanediol) of female hormones (estrogen, progesterone) may be used.

Embodiments of the present invention have been described above. However, the present invention is not limited to the above description. Those skilled in the art can modify the above embodiments as appropriate, and such modifications are also encompassed within the scope of the present invention as long as they include the features of the present invention. For example, the shape, dimension, material, arrangement, installation form, and the like of each element included in the female life stage prediction system are not limited to those illustrated and can be modified as appropriate.

Furthermore, the elements in each of the above embodiments can be combined with each other as long as technically feasible. Such combinations are also encompassed within the scope of the present invention as long as they include the features of the present invention.

What is claimed is:

1. A female life stage prediction method comprising:
measuring a secretion amount of a hormone of a user, based on at least any of bodily fluid discharged to an outside of a body of the user or a volatile component of the bodily fluid;
recording the secretion amount measured;
estimating a fluctuation in the secretion amount of the hormone of the user, based on the secretion amount recorded;
making a prediction about a time of at least any of menarche, menstruation, or menopause of the user, based on the fluctuation estimated; and
issuing a notification of a result of the prediction.

2. A female life stage prediction system comprising:
a measurement unit configured to measure a secretion amount of a hormone of a user, based on at least any of bodily fluid discharged to an outside of a body of the user or a volatile component of the bodily fluid;
a recording unit configured to record the secretion amount measured;
a hormone secretion amount fluctuation estimation unit configured to estimate a fluctuation in the secretion amount of the hormone of the user, based on the secretion amount recorded;
a life stage prediction unit configured to make a prediction about a time of at least any of menarche, menstruation, or menopause of the user, based on the fluctuation estimated; and
an output unit configured to output a result of the prediction.

3. The female life stage prediction system according to claim 2, wherein
the bodily fluid is sweat, and
the measurement unit includes a detection unit installed on a seat face of a toilet seat and configured to detect the hormone contained in the sweat or in a volatile component of the sweat.

4. The female life stage prediction system according to claim 2, wherein
the bodily fluid is urine, and
the measurement unit includes a detection unit configured to detect, from air in an inner toilet bowl, the hormone contained in a volatile component of the urine.

5. The female life stage prediction system according to claim 3, further comprising:
a seating sensor configured to detect a person sitting on a toilet seat, wherein
the measurement unit starts measurement when the seating sensor detects that the user is seated on the toilet seat.

6. The female life stage prediction system according to claim 2, wherein
the life stage prediction unit predicts an onset of menarche when the fluctuation of the hormone estimated satisfies a predetermined condition.

7. The female life stage prediction system according to claim 6, wherein
the life stage prediction unit predicts a time of next menstruation, based on the fluctuation of the hormone estimated, the fluctuation being cyclical.

8. The female life stage prediction system according to claim 7, further comprising:
an imaging unit configured to capture an image of a surface of an inner toilet bowl, wherein
the life stage prediction unit predicts a time of menstruation, based on a time of actual menstruation determined based on the image captured by the imaging unit and the cyclical fluctuation of the hormone estimated.

9. The female life stage prediction system according to claim 2, further comprising:
a health state prediction unit configured to calculate a stress level of the user, wherein
the stress level is stored in association with the secretion amount of the hormone measured when the stress level is calculated, and
the health state prediction unit predicts an onset of premenstrual syndrome, based on a relationship between the stress level stored and the secretion amount of the hormone associated with the stress level, and a current secretion amount of the hormone.

10. The female life stage prediction system according to claim 2, further comprising:
a sensor configured to acquire blood flow information of the user; and
a health state prediction unit configured to predict a possibility of anemia of the user, based on the blood flow information and the fluctuation of the hormone estimated.

11. The female life stage prediction system according to claim 2, wherein
the life stage prediction unit predicts that menopause is approaching, based on a decreasing trend of the secretion amount of the hormone.

12. The female life stage prediction system according to claim 2, further comprising:
a controller configured to suggest an action to the user, based on the time of menstruation predicted and calendar information.

13. The female life stage prediction system according to claim 2, wherein
a fluctuation state of the secretion amount of the hormone of the user is transmitted to a server, and
the server makes a comparison between the fluctuation state and a hormone secretion amount fluctuation model, and outputs menstrual state information of the user based on a result of the comparison.

14. A female life stage prediction program for causing a computer to execute processing comprising:
estimating, based on a secretion amount of a hormone of a user measured based on at least any of bodily fluid discharged to an outside of a body of the user or a volatile component of the bodily fluid, and stored in a recording unit, a fluctuation in the secretion amount of the hormone of the user;
making a prediction about a time of at least any of menarche, menstruation, or menopause of the user, based on the fluctuation estimated; and
outputting a result of the prediction.
